(19) United States
(12) Patent Application Publication
(10) Pub. No.: US 2023/0002781 A1
(43) Pub. Date: Jan. 5, 2023

(54) Methods and Compositions for Modifying Plant Immunity

(71) Applicant: The Regents of the University of California, Oakland, CA (US)

(72) Inventors: Ksenia Vladimirovna Krasileva, Berkeley, CA (US); Daniil M. Prigozhin, Berkeley, CA (US); Brian J. Staskawicz, Berkeley, CA (US); Furong Liu, Berkeley, CA (US)

(21) Appl. No.: 17/781,229

(22) PCT Filed: Dec. 4, 2020
(86) PCT No.: PCT/US2020/063203

§ 371 Date: May 31, 2022

Related U.S. Application Data
(60) Provisional application No. 62/943,404, filed on Dec. 4, 2019.

(51) Int. Cl. C12N 15/82

(52) U.S. Cl. CPC C12N 15/8279

(57) ABSTRACT

Disclosed herein are methods for identifying amino acid residues of NLRs that are likely involved in pathogen immunity. Also disclosed are methods for modifying plant immunity which comprise modifying (e.g., substituting, deleting, or adding an amino acid before or after) one or more of the amino acid residues that have been identified as being likely involved in pathogen immunity.

### CROSS-REFERENCE TO RELATED APPLICATIONS

This application claims the benefit of U.S. Patent Application No. 62/943,404, filed Dec. 4, 2019, which is herein incorporated by reference in its entirety.

### REFERENCE TO A SEQUENCE LISTING SUBMITTED VIA EFS-WEB

The content of the ASCII text file of the sequence listing named “20201204_034044_213WO1_ST25” which is 18.7 kb in size was created on Nov. 29, 2020 and electronically submitted via EFS-Web herewith the application is incorporated herein by reference in its entirety.

### BACKGROUND OF THE INVENTION

### 1. Field of the Invention

The field generally relates to recombinant plant immune receptors.

### 2. Description of the Related Art

Plant pathogens threaten production of valuable crops. Identification of genetic sources of resistance to pathogens is an active area of both academic and industry-based research and has potential to increase yields and reduce reliance on environmentally damaging chemicals used to control infections. Current methods for finding new resistant plant varieties rely on either naturally occurring immune receptors that need to be crossed into elite varieties or on induced variation produced by random mutagenesis of the whole genome that requires back-crosses to remove unwanted variation. In case of finding new natural variants, traditional approaches rely on genetic mapping and Mendelian genetics, which can take over 5 years even when newest sequencing technologies are used. There is no guarantee that after this process, the identified gene will work on its own in a new variety or how durable it will be. Another way of finding new natural sources of disease resistance is through genome wide association mapping; however, this process will only identify genes if their prevalence in natural populations is above 5% and will miss any rare variants that are key to generation of new pathogen recognition specificities. Finally, new sources of disease resistance can be identified by introducing genetic variation by mutagenesis; this involves generation of a mutagenized population, screening it, isolating the region of interest and then breeding out background mutations, a process that can take 4-6 years and often the causative mutation responsible for the phenotype is not mapped beyond a general genomic region.

Plant immunity is encoded in the germline, a feature plants share with the majority of eukaryotes. Plants deploy hundreds of immune receptors to detect presence of pathogen-derived molecules and elicit an immune response. At the same time, plant pathogens—viruses, bacteria, and fungi—evolve rapidly to evade immune recognition. The two main classes of plant immune receptors include transmembrane receptor like kinases and pattern recognition receptors that monitor the extracellular environment, and the intracellular Nucleotide Binding Leucine Rich Repeat receptors (NLRs) that can detect pathogen-derived molecules or their activities inside the plant cells. Variation in these receptors on a population level provides the necessary arsenal of pathogen recognition specificities.

Plant NLRs typically comprise three domains: a central Nucleotide Binding (NB-ARC) domain that mediates receptor oligomerization upon activation, a signaling domain, e.g., an N-terminal Toll Interleukin 1 like Receptor (TIR) or Coiled Coil (CC) domain, and the C-terminal Leucine Rich Repeat (LRR) domain that mediates both activating and self-inhibitory protein-protein interactions. NLRs have been shown to recognize pathogens using three main modes. First, direct binding to the pathogen-derived effector molecules with binding specificity commonly encoded in the LRR domain. Second, the indirect recognition of effector activities on other plant proteins, with the NLRs guarding plant proteins targeted by effectors. Third, indirect recognition of modifications to a non-canonical integrated domain of the NLR, which acts as a bait for the effector.

### SUMMARY OF THE INVENTION

### Identification Methods

In some embodiments, the present invention is directed to a method of identifying one or more amino acid residues in a plant immunity receptor that are likely involved in binding its cognate ligand, which comprises determining the Shannon entropy of each amino acid of the receptor; selecting an entropy cutoff value of at least 1.0, at least 1.1, at least 1.2, at least 1.3, at least 1.4, at least 1.5, at least 1.6, at least 1.7, at least 1.8, at least 1.9, or at least 2.0; and identifying at least one amino acid residue among the amino acid residues having the highest entropy values above the entropy cutoff value. In some embodiments, the entropy cutoff value is at least 1.5. In some embodiments, the Shannon entropy is determined from a plurality of amino acid sequences from homologs of the plant immunity receptor, and the plant immunity receptor and the homologs are of the same plant species. In some embodiments, the plurality of amino acid sequences comprises at least 25, at least 30, at least 35, at least 40, at least 45, at least 50, at least 55, at least 60, at least 65, at least 70, at least 75, or at least 80 amino acid sequences. In some embodiments, the plants are genetically diverse. In some embodiments, at least ten amino acid residues having the highest entropy values are identified as being likely involved in binding the cognate ligand. In some embodiments, 5-20, 6-19, 7-18, 8-16, 9-16, or 10-15 amino acid residues having the highest entropy values are identified as being likely involved in binding the cognate ligand. In some embodiments, the method further comprises determining the hydrophobicity of each amino acid of the receptor and identifying one or more of the amino acid residues having the highest entropy values and the highest hydrophobicity as being likely involved in binding the cognate ligand.

### Modification Methods

In some embodiments, the present invention is directed to a method of modifying pathogen recognition, binding, and/or specificity of a plant immunity receptor, which comprises modifying one or more amino acid residues that are identified as being involved in binding the cognate ligand of the plant immunity receptor as described herein, e.g., “Identification Methods” above. In some embodiments, the one or more amino acid residues are substituted with a different amino acid. In some embodiments, a region of contiguous amino acid residues comprising the one or more amino acid residues is substituted with a region of contiguous amino acid residues obtained from a different plant immunity receptor.

In some embodiments, the present invention is directed to a method of modifying a Sr33 plant immunity receptor, which comprises modifying one or more of the amino acid residues that correspond to amino acid residue positions 703-887 of SEQ ID NO: 1. In some embodiments, the amino acid residue positions are selected from (a) 707, 709, 710, 735, 737, 738, 765, 767, 768, 793, 795, 796, 818, 820, and 821 of SEQ ID NO: 1, or (b) 707, 709, 711, 735, 737, 741, 765, 767, 793, 795, 796, 818, 820, 821, 832, 841, 843, 864, 866, and 880 of SEQ ID NO: 1. In some embodiments, the Sr33 plant immunity receptor comprises at least 90%, at least 91%, at least 92%, at least 93%, at least 94%, at least 95%, at least 96%, at least 97%, at least 98%, at least 99%, or 100% sequence identity to SEQ ID NO: 1.

In some embodiments, the present invention is directed to a method of modifying a plant immunity receptor of Arabidopsis thaliana, which comprises modifying one or more amino acid residues of the plant immunity receptor that correspond the highly variable amino acid residues as set forth in Table 1 or Table 2.

### Products

In some embodiments, the present invention is directed to a plant immunity receptor made or modified as described herein, e.g., “Modification Methods” above. In some embodiments, the present invention is directed to a nucleic acid molecule that encodes the plant immunity receptor. In some embodiments, the present invention is directed to a vector which comprises the nucleic acid molecule. In some embodiments, the present invention is directed to a cell which comprises the vector. In some embodiments, the present invention is directed to a plant which comprises the plant immunity receptor. In some embodiments, the present invention is directed to a plant which comprises the cell that comprises the vector. In some embodiments, the present invention is directed to a population of plants comprising a plurality of plants which comprise the plant immunity receptor and/or the cell that comprises the vector.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 shows the entropy and hydrophobicity of LRRs in Sr33 and amino acid modifications that may be made to modify the pathogen recognition, specificity, and/or binding of the receptor.

FIG. 2 shows that the chimeric receptor Sr3350 (comprising Sr50 leucine-rich repeats residues 8 to 14 grafted onto Sr33) recognizes the pathogen effector AvrSr50. Phenotypes of the hypersensitive response (grey) are visible in the leaves of Nicotiana benthamiana (N. benthamiana) with Agrobacterium-mediated transient expression. Top: Transient expression in N. benthamiana of Sr50, Sr33, Sr3350, AvrSr50, or AvrSr50QCMJC alone. Middle: Transient expression of in N. benthamiana of Sr50, Sr33, Sr3350 with the pathogen effector AvrSr50. Bottom: Transient expression of in N. benthamiana of Sr50, Sr33, Sr3350 with AvrSr50 variant race AvrSr50QCMJC found in Puccinia graminis f. sp. tritici. The binary vector p1776 containing each gene was transformed into Agrobacterium strain GV3101 and inoculated in 4-5 week-old N. benthamiana plants at an OD₆₀₀ of 0.025 for each. Four experimental repeats were performed for each experiment on different plants. The images were taken two days post infection.

FIG. 3 shows allelic swap and cell death assays with AvrSr50 in Triticum aestivum (wheat) protoplasts. The y axis represents luminescence from the luciferase reporter designating live transfected cells, lack of luminescence indicates cell death response. The x axis shows different combinations of constructs tested in wheat protoplasts. N=4 biological replicates, each containing 3 technical replicates. **Paired t-test<0.0001.

FIG. 4 shows the entropy and hydrophobicity of LRRs in RPP1 of A. thaliana, and amino acid modifications that may be made to modify the pathogen recognition, specificity, and/or binding of the receptor.

FIG. 5, FIG. 6, and FIG. 7 show the entropy and hydrophobicity of LRRs in RPP13 of A. thaliana, and amino acid modifications that may be made to modify the pathogen recognition, specificity, and/or binding of the receptor.

### DETAILED DESCRIPTION OF THE INVENTION

Disclosed herein are methods for identifying amino acid residues of NLRs that are likely involved in pathogen immunity. Also disclosed are methods for modifying plant immunity which comprise modifying (e.g., substituting, deleting, or adding an amino acid before or after) one or more of the amino acid residues that have been identified as being likely involved in pathogen immunity.

As disclosed herein, Shannon entropy analysis was used to NLRs to identify amino acid residues in NLRs that are likely involved in pathogen recognition, specificity, and/or binding. See Stewart, et al. (1997) Molec Immunol 34(15): 1067-1082. Shannon entropy is given by the formula:

$$H = - {\sum }_{i = 1}^{20} {p}_{i} {log}_{2} {p}_{i}$$

where pi is the fraction of one of the twenty amino acids in a column of a protein sequence alignment. Shannon entropy calculates the amount of variability at each column, i.e., amino acid position, of a plurality of aligned sequences. Amino acid positions that little amino acid variability have low entropy and amino acid positions that have high amino acid variability have high entropy.

Briefly, the amino acid sequences of a given receptor in at least 60 genetically diverse plants in initial studies (and at least 25 genetically diverse plants in later studies) within a given species were aligned and then the Shannon entropy at each amino acid position was determined. In initial studies, if the given receptor had 15 or more amino acid residues with entropy above a cutoff of 1.5, the receptor was classed as highly variable and the top 10-15 amino acids with highest entropy were found to be involved in pathogen recognition, specificity, and/or binding. In later studies, if the given receptor had 15 or more amino acid residues with entropy above a cutoff of 1.5, the receptor was classed as highly variable and amino acids entropy above a cutoff of 1.5 were found to be involved in pathogen recognition, specificity, and/or binding.

### Shannon Entropy to Identify Highly Variable Residues in Immune Receptors

To test if Shannon entropy can serve as a valid measure to detect specificity-determining residues in immune receptors, we tested it on two well studied systems: human antibody and the variable leukocyte receptor from lampreys. In the case of the antibody, we restricted our analysis to the Fab fragment of a known IgG antibody. By aligning sequences of homologs of the IgG antibody as deposited in RCSB Protein Data Bank (rcsb.org) for both heavy and light chains, and calculating the entropy scores for individual positions, we observed that residues with the highest variability scores occurred in the N-termini of both the heavy and light chains and coincided with their complementarity determining regions (CDRs). Similar analysis of the lamprey VLR, the receptor molecule in the lamprey adaptive immune system, highlighted the concave side of the leucine rich repeat domain as the location for the most diverse surface residues in these receptors. These results are consistent with information of the binding sites as provided in the art.

### Sr33/Sr50 Receptor Family

### Sr33/Sr50 Binding Site Prediction

To confirm that Shannon entropy analysis may be used to identify the sites in plant immunity receptors that are likely involved in pathogen recognition and binding, the Sr33/Sr50 family of receptors from Triticiea was used because its members directly bind a diverse array of pathogens. BLAST sequence searches of publicly available databases were performed to select about 100 homologs of Sr50. The selected sequences were aligned and the entropy scores of each aligned amino acid residue was calculated using methods in the art. Specifically, the alignments were filtered to exclude outliers using R and OD-seq (Jehl, et al. 2015). The filtered alignments were scored using a Shannon's entropy function (Shannon & Weaver 1949) that generates a numerical score for each position in the alignment.

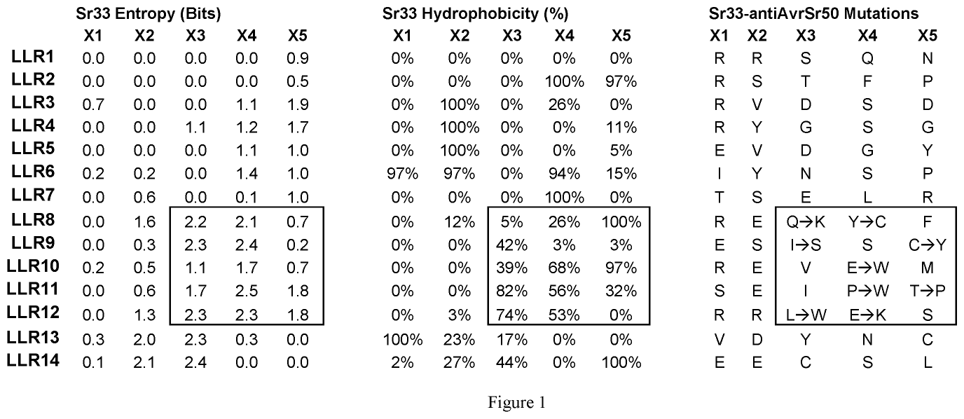

Aligned amino acid residues that consistently exceed a score cutoff of 1.5 bits were classed as highly variable and were hypothesized as being involved in the binding and recognition of pathogen molecules. The highly variable residues were cross-checked against surface prediction of the LRR domain and hydrophobicity. Sets of 10-15 amino acid residues that were predicted to cluster on the surface of the receptor were investigated for presence of exposed aromatic and hydrophobic residues in the natural sequences. Clusters that show high variability (entropy scores) and a core of exposed hydrophobic residues are considered as candidate specificity-determining regions of the plant immune receptor. FIG. 1 shows the entropy and hydrophobicity of LRRs in Sr33 and amino acid modifications that may be made to modify the pathogen recognition, specificity, and/or binding of the receptor.

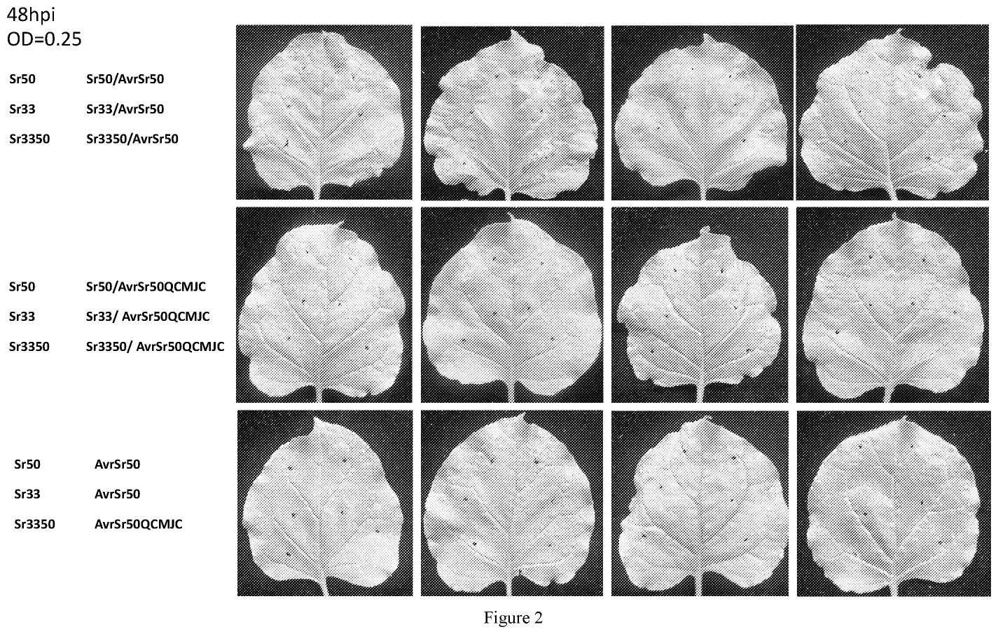

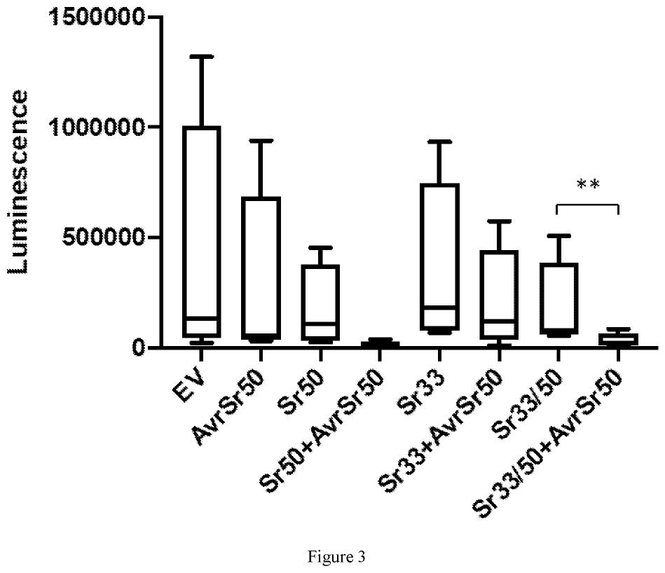

Similarly, the LRR domain of Sr33 (amino acid residues 703-887 of AGQ17390.1) was determined. Using methods in the art, the LRR domain of Sr33 was substituted with the LRR domain of Sr50 (amino acid residues 707-891 of ALO61074.1) to result in a chimeric receptor, “Sr3350”. Using a co-infiltration assay in N. benthamiana known in the art, the chimeric receptor Sr3350 was found to recognize the cognate target of Sr50, i.e., AvrSr50. See FIG. 2. Using protoplast transfection assay in Triticum aestivum known in the art, the chimeric receptor Sr3350 was found to recognize AvrSr50, the co-transfection of Sr3350 and AvrSr50 produced statistically significant more protoplast cell death (Paired t-test<0.0001) than transfection of Sr3350 alone. See FIG. 3.

These experiments indicate Shannon entropy analysis may be used to identify one or more amino acid residues in a plant immune receptor which are involved in pathogen recognition and specificity, and which may be modified to change the pathogen recognition and specificity of the receptors. For example, amino acid residues 707, 709, 710, 735, 737, 738, 765, 767, 768, 793, 795, 796, 818, 820, and 821 of Sr33 were identified as being highly variable. Using a strict 1.5 bit score cutoff, amino acid residues 707, 709, 711, 735, 737, 741, 765, 767, 793, 795, 796, 818, 820, 821, 832, 841, 843, 864, 866, and 880 of Sr33 were identified as being highly variable. As such, the pathogen recognition, specificity, and/or binding of Sr33 may be modified by modifying one or more of these residues in any combination. In some embodiments, one highly variable amino acid residue is modified. In some embodiments, two or more highly variable amino acid residues are modified. In some embodiments, three or more highly variable amino acid residues are modified. In some embodiments, four or more highly variable amino acid residues are modified. In some embodiments, five or more highly variable amino acid residues are modified. In some embodiments, six or more highly variable amino acid residues are modified. In some embodiments, seven or more highly variable amino acid residues are modified. In some embodiments, eight or more highly variable amino acid residues are modified. In some embodiments, nine or more highly variable amino acid residues are modified. In some embodiments, ten or more or more highly variable amino acid residues are modified.

### Highly Variable NLRs in Arabidopsis Thaliana

Phylogenetic analysis was used to group the published NLRome into clades that were large enough to preserve the variability signal while being amenable to full-length de novo alignment. Shannon entropy was used to collapse the sequence alignments into simple 2D graphs while highlighting areas of increased diversity. The annotation of individual Leucine Rich Repeat motifs in the LRR domain combined with the variability analysis highlighted amino acid residues in the LRR domain that are likely involved in pathogen recognition specificity.

The results herein show that around 30% of all Arabidopsis NLRs belong to rapidly diversifying families and are therefore candidates for direct recognition. These are distributed in the NLR phylogeny among both CC- and TIR-dependent NLRs. The results further show that, in the twelve clades that contain highly variably NLRs (hvNLRs), the highly variable residues are positioned near each other in a “cluster” on the surface of the NLRs, but the locations of the clusters are not conserved between different families of NLRs.

### Arabidopsis NLRome Shows Variable Rates of NLR Diversification

Unlike the receptors of animal adaptive immunity such as the antibody, the T-cell receptor, and the lamprey VLR, plant immune receptor specificities are germline encoded. To carry out variability analyses on NLR clades of A. thaliana, we identified groups of closely related NLR's within the overall NLR phylogeny that were close enough in sequence to produce high quality alignments, yet large enough to preserve the variability signal using methods in the art. See, e.g., Bailey et al. First, an overall NLR phylogeny was constructed on the basis of only the NB-ARC domain, and clades with strong bootstrapping were identified. Second, the full-length sequences of identified clades were optimally aligned, and the alignments were evaluated by Shannon entropy analysis and assigned entropy scores. Clades that produced high variability scores were marked for further splitting on the basis of phylogenetic trees built on the full-length alignment. Because exclusion of more distantly related sequences improves alignment, the process was repeated for several subclades to produce a final clade assignment, in which most clades had very low average entropy scores and most contained 70 or fewer members, with few containing more than one gene from a given ecotype.

The highly variable amino acid residues identified in the highly variable genes of Arabidopsis thaliana as described herein are provided in Table 1 and Table 2.

TABLE 1
Highly variable immune genes from Arabidopsis thaliana

| | | NB-ARC | |
|---|---|---|---|
| Accession No. | Uniprot No. | Residues | Highly Variable Amino Acid Residues |
| AT1G31540.1 | F4I9F1_ARATH | 183-454 | 636, 637, 659, 660, 662, 683, 684, 704, 706, 707, 727, 728, 741, 745, 751 |
| AT1G31540.2 | | 183-454 | 636, 659, 684, 706, 707, 727, 780, 782, 802, 806, 807, 869, 1008, 1035, 1037 |
| AT1G58602.1 | DRL9_ARATH | 163-511 | 735, 737, 761, 808, 832, 856, 857, 879, 923, 968, 970, 995, 996, 1014, 1040 |
| AT1G58602.2 | | 163-511 | 735, 737, 761, 808, 832, 856, 857, 879, 923, 968, 970, 995, 996, 1014, 1040 |
| AT1G58807.1 | DRL10_ARATH | 164-507 | 565, 634, 635, 659, 729, 802, 826, 848, 850, 873, 874, 895, 923, 944, 968 |
| AT1G58807.2 | | 164-507 | 565, 634, 635, 659, 677, 727, 729, 731, 755, 801, 802, 826, 848, 850, 852 |
| AT1G58848.1 | DRL11_ARATH | 164-507 | 643, 644, 667, 737, 810, 834, 858, 859, 881, 882, 928, 930, 973, 975, 1021 |
| AT1G58848.2 | | 164-507 | 643, 644, 667, 737, 810, 834, 858, 859, 881, 882, 928, 930, 973, 975, 1021 |
| AT1G59124.1 | DRL44_ARATH | 164-507 | 565, 634, 635, 659, 677, 727, 729, 731, 755, 801, 802, 826, 848, 850, 852 |
| AT1G59218.1 | DRL45_ARATH | 164-507 | 643, 644, 667, 737, 810, 834, 858, 859, 881, 882, 928, 930, 973, 975, 1021 |
| AT1G59218.2 | | 164-507 | 643, 644, 667, 737, 810, 834, 858, 859, 881, 882, 928, 930, 973, 975, 1021 |
| AT1G61180.1 | Q2V4G0_ARATH | 155-482 | 419, 467, 520, 542, 590, 615, 679, 681, 742, 744, 763, 765, 767, 821, 823 |
| AT1G61180.2 | | 155-482 | 419, 467, 520, 542, 590, 615, 679, 681, 742, 744, 763, 765, 767, 821, 823 |
| AT1G61190.1 | DRL16_ARATH | 156-486 | 420, 425, 524, 546, 594, 619, 683, 685, 748, 750, 769, 771, 773, 828, 830 |
| AT1G61300.1 | DRL17_ARATH | 44-371 | 308, 356, 409, 431, 479, 504, 567, 569, 630, 632, 651, 653, 655, 709, 711 |
| AT1G61310.1 | DRL18_ARATH | 157-495 | 421, 480, 533, 555, 603, 628, 692, 694, 755, 757, 776, 778, 780, 834, 836 |
| AT1G62630.1 | DRL14_ARATH | 153-486 | 472, 557, 581, 626, 700, 723, 725, 726, 747, 772, 773, 828, 891, 892, 893 |
| AT1G63360.1 | DRL20_ARATH | 153-486 | 472, 555, 579, 624, 698, 721, 723, 724, 745, 770, 771, 802, 826, 883, 884 |
| AT1G69550.1 | F4I270_ARATH | 255-517 | 678, 679, 720, 792, 796, 816, 820, 840, 844, 864, 890, 893, 911, 938, 940 |
| AT2G14080.1 | F4IFF6_ARATH | 230-495 | 581, 637, 659, 660, 680, 683, 754, 778, 797, 825, 826, 959, 1150, 1153, 1155 |
| AT3G44400.1 | Q9M285_ARATH | 214-483 | 472, 509, 634, 656, 681, 725, 815, 882, 937, 938, 939, 941, 943, 962, 1007 |
| AT3G44400.2 | | 214-483 | 472, 509, 634, 656, 681, 725, 815, 882, 937, 938, 939, 941, 943, 962, 1007 |
| AT3G44480.1 | RPP1_ARATH | 273-542 | 531, 568, 678, 769, 835, 859, 862, 882, 927, 970, 1091, 1092, 1093, 1109, 1150 |
| AT3G44630.1 | F4J359_ARATH | 269-597 | 527, 591, 703, 771, 884, 887, 907, 952, 996, 1063, 1118, 1119, 1124, 1172, 1173 |
| AT3G44630.2 | | 269-597 | 527, 591, 703, 771, 884, 887, 907, 952, 996, 1063, 1118, 1119, 1124, 1172, 1173 |
| AT3G44630.3 | | 269-597 | 527, 591, 703, 771, 884, 887, 907, 952, 996, 1063, 1118, 1119, 1124, 1172, 1173 |
| AT3G44670.1 | F4J361_ARATH | 269-530 | 527, 570, 653, 680, 771, 883, 886, 906, 939, 1006, 1061, 1064, 1071, 1091, 1125 |
| AT3G44670.2 | | 269-530 | 527, 570, 653, 680, 771, 883, 886, 906, 939, 1006, 1061, 1064, 1071, 1091, 1125 |
| AT3G46530.1 | RPP13_ARATH | 163-498 | 553, 554, 573, 619, 620, 622, 624, 684, 712, 788, 792, 811, 813, 814, 815 |
| AT4G16860.1 | RPP4_ARATH | 185-446 | 426, 461, 473, 529, 531, 575, 594, 638, 661, 775, 776, 798, 818, 954, 1000 |
| AT4G16890.1 | SNC1_ARATH | 183-454 | 424, 459, 471, 507, 527, 528, 529, 558, 574, 577, 621, 644, 800, 1003, 1070 |
| AT4G16920.1 | Q9SUK4_ARATH | 181-442 | 422, 457, 469, 525, 527, 558, 577, 621, 622, 644, 664, 756, 781, 782, 937 |
| AT4G16940.1 | F4JNB6_ARATH | 143-403 | 384, 391, 423, 435, 471, 487, 549, 594, 596, 597, 687, 748, 750, 903, 906 |
| AT4G16950.1 | RPP5_ARATH | 188-453 | 463, 475, 531, 533, 564, 583, 627, 650, 764, 787, 788, 989, 1079, 1198, 1263 |
| AT4G16950.2 | | 188-453 | 463, 475, 531, 533, 564, 583, 627, 650, 764, 787, 788, 989, 1079, 1198, 1263 |
| AT4G16960.1 | O23536_ARATH | 187-447 | 96, 428, 435, 467, 479, 515, 569, 614, 616, 617, 705, 766, 768, 921, 924 |
| AT5G38350.1 | Q9FF28_ARATH | 26-290 | 291, 387, 461, 463, 505, 509, 523, 529, 531, 620, 661, 684, 686, 788, 816 |
| AT5G41740.1 | F4JYI4_ARATH | 176-439 | 576, 620, 623, 641, 643, 644, 646, 729, 754, 794, 815, 840, 908, 914, 1010 |
| AT5G41740.2 | | 176-439 | 576, 620, 623, 641, 643, 644, 646, 729, 754, 794, 815, 840, 908, 914, 1010 |
| AT5G41750.1 | Q9LSX5_ARATH | 186-447 | 590, 634, 637, 655, 657, 658, 660, 743, 767, 807, 828, 853, 921, 927, 1068 |
| AT5G41750.2 | | 186-447 | 590, 634, 637, 655, 657, 658, 660, 743, 767, 807, 828, 853, 921, 927, 1068 |
| AT5G43470.1 | RPP8_ARATH | 165-502 | 443, 485, 486, 610, 632, 678, 698, 776, 778, 779, 800, 802, 803, 804, 825 |
| AT5G43470.2 | | 165-502 | 443, 485, 486, 610, 632, 678, 698, 776, 778, 779, 800, 802, 803, 804, 825 |
| AT5G43740.1 | DRL33_ARATH | 155-481 | 519, 521, 545, 568, 590, 615, 639, 688, 712, 774, 776, 777, 809, 813, 814 |
| AT5G43740.2 | | 155-481 | 519, 521, 545, 568, 590, 615, 639, 688, 712, 774, 776, 777, 809, 813, 814 |
| AT5G46490.2 | | 183-452 | 225, 265, 274, 284, 465, 518, 536, 563, 646, 663, 706, 723, 725, 746, 749 |
| AT5G46510.1 | Q9FHF4_ARATH | 184-455 | 102, 126, 565, 710, 724, 746, 799, 801, 873, 1015, 1020, 1039, 1041, 1083, 1085 |
| AT5G46520.1 | VICTR_ARATH | 185-456 | 103, 127, 566, 711, 725, 747, 800, 802, 874, 1016, 1021, 1040, 1042, 1084, 1086 |
| AT5G48620.1 | RP8L4_ARATH | 165-502 | 443, 485, 486, 610, 632, 678, 698, 776, 778, 779, 800, 802, 803, 804, 825 |

Amino acid residues in the NB-ARC domain are underlined and the remaining residues are located in the LRR region.

TABLE 2
Highly variable immune genes from Arabidopsis thaliana

| | Common | | | | | |
|---|---|---|---|---|---|---|
| Gene | Name | preNB | NB-ARC | linker | LRR | post-LRR |
| AT1G31540.2 | RPP9 | | | | 636, 659, 660, 662, 683, 684, | 1006, 1008, 1009, |
| | | | | | 706, 707, 727, 728, 778, 780, | 1011, 1035, 1036, |
| | | | | | 782, 783, 802, 804, 806, 807, | 1037, 1038, 1044, |
| | | | | | 829, 846, 851, 869, 897 | 1046 |
| AT1G58602.1 | RPP7 | | | 570 | 616, 638, 643, 665, 735, 737, | |
| | | | | | 759, 761, 784, 807, 808, 809, | |
| | | | | | 832, 856, 857, 879, 887, 923, | |
| | | | | | 949, 968, 970, 971, 993, 995, | |
| | | | | | 996, 1014, 1016, 1040, 1062, | |
| | | | | | 1064, 1065, 1085, 1087 | |
| AT1G58807.1 | | 49 | | | 565, 566, 635, 636, 638, 659, | 996 |
| | | | | | 677, 727, 729, 731, 755, 801, | |
| | | | | | 802, 826, 848, 850, 851, 861, | |
| | | | | | 873, 874, 895, 897, 918, 922, | |
| | | | | | 923, 944, 946, 947, 968 | |
| AT1G58848.1 | | 49 | | | 573, 576, 641, 642, 644, 667, | |
| | | | | | 685, 735, 737, 739, 763, 809, | |
| | | | | | 810, 834, 856, 858, 859, 869, | |
| | | | | | 881, 882, 903, 905, 907, 910, | |
| | | | | | 928, 930, 952, 954, 955, 971, | |
| | | | | | 975, 976, 997, 999, 1000, 1021 | |
| AT1G59124.1 | | 49 | | | 565, 566, 635, 636, 638, 659, | |
| | | | | | 677, 727, 729, 731, 755, 801, | |
| | | | | | 802, 826, 848, 850, 851 | |
| AT1G59218.1 | | 49 | | | 573, 576, 641, 642, 644, 667, | 1049 |
| | | | | | 685, 735, 737, 739, 763, 809, | |
| | | | | | 810, 834, 856, 858, 859, 869, | |
| | | | | | 881, 882, 903, 905, 907, 910, | |
| | | | | | 928, 930, 952, 954, 955, 971, | |
| | | | | | 975, 976, 997, 999, 1000, 1021 | |
| AT1G61180.1 | | 15, 19 | 418, 419, 421, | | 520, 522, 542, 566, 568, 590, | |
| | | | 424, 465, 467, | | 612, 613, 615, 636, 659, 679, | |
| | | | 468 | | 681, 683, 703, 705, 707, 722, | |
| | | | | | 740, 742, 744, 745, 763, 765, | |
| | | | | | 767, 795, 797, 799, 800, 819, | |
| | | | | | 821, 823, 824, 847, 850 | |
| AT1G61190.1 | RPP39 | 16, 20 | 419, 420, 422, | | 524, 526, 546, 570, 572, 594, | |
| | | | 425 | | 616, 617, 619, 640, 663, 683, | |
| | | | | | 685, 687, 707, 709, 711, 728, | |
| | | | | | 746, 748, 750, 751, 769, 771, | |
| | | | | | 773, 802, 804, 806, 807, 826, | |
| | | | | | 828, 830, 831, 854, 856, 897 | |
| AT1G61300.1 | | 17, 21 | 307, 308, 310, | | 409, 411, 431, 455, 457, 479, | |
| | | | 313, 354, 356, | | 501, 502, 504, 525, 548, 567, | |
| | | | 357 | | 569, 571, 591, 593, 595, 610, | |
| | | | | | 628, 630, 632, 633, 651, 653, | |
| | | | | | 655, 683, 685, 687, 688, 707, | |
| | | | | | 709, 711, 712, 735, 737 | |
| AT1G61310.1 | | 17, 21 | 420, 421, 423, | | 533, 535, 555, 579, 581, 603, | |
| | | | 426, 467, 469, | | 625, 626, 628, 649, 672, 692, | |
| | | | 481 | | 694, 696, 716, 718, 720, 735, | |
| | | | | | 753, 755, 757, 758, 776, 778, | |
| | | | | | 780, 808, 810, 812, 813, 832, | |
| | | | | | 834, 836, 837, 860, 862 | |
| AT1G62630.1 | | | 271, 467, 468, | | 546, 548, 557, 579, 581, 603, | 855, 881 |
| | | | 472 | | 605, 626, 700, 705, 723, 725, | |
| | | | | | 726, 747, 750, 772, 773, 791, | |
| | | | | | 804, 806, 807, 828, 830 | |
| AT1G69550.1 | | | 549, 550 | 631, 633 | 678, 679, 693, 701, 704, 720, | 1341 |
| | | | | | 722, 725, 743, 748, 772, 791, | |
| | | | | | 792, 796, 797, 815, 816, 818, | |
| | | | | | 820, 840, 842, 844, 863, 864, | |
| | | | | | 865, 866, 868, 869, 887, 892, | |
| | | | | | 893, 911, 912, 914, 916, 936, | |
| | | | | | 941, 960, 962, 965, 989, 1032, | |
| | | | | | 1056, 1060, 1061, 1080, 1082, | |
| | | | | | 1084, 1085, 1126, 1127, 1129, | |
| | | | | | 1132, 1150, 1155, 1220, 1222 | |
| AT2G14080.1 | RPP28 | 177 | | 558 | 581, 637, 659, 660, 680, 682, | 1128, 1150, 1153, |
| | | | | | 683, 754, 778, 797, 801, 802, | 1155, 1214 |
| | | | | | 821, 849, 852, 868, 930, 959 | |
| AT3G44400.1 | | 8, 191 | 223, 472, 507, | 565, 566, 581 | 588, 591, 592, 594, 610, 613, | 856, 858, 860, |
| | | | 509 | | 633, 634, 656, 679, 681, 701, | 881, 882, 883, |
| | | | | | 702, 725, 726, 748, 767, 771, | 884, 935, 937, |
| | | | | | 790, 815, 817, 835 | 938, 939, 941, |
| | | | | | | 943, 960, 961, |
| | | | | | | 962, 994, 1004 |
| AT3G44480.1 | RPP1 | 4, 65, 241 | 282, 531, 563, | 624, 625, 640 | 647, 649, 651, 677, 678, 700, | 1011, 1013, 1015, |
| | | | 564, 566, 568 | | 723, 725, 745, 746, 769, 770, | 1034, 1035, 1036, |
| | | | | | 787, 790, 792, 811, 812, 835, | 1089, 1091, 1093, |
| | | | | | 836, 859, 860, 862, 864, 882, | 1095, 1097, 1148, |
| | | | | | 903, 904, 906, 908, 927, 948, | 1149, 1150, 1151, |
| | | | | | 950, 953, 970, 972, 990 | 1187, 1188 |
| AT3G44630.1 | | 4, 56, 237 | 278, 527, 560, | 647, 648, 663 | 670, 672, 673, 674, 702, 703, | 1037, 1039, 1041, |
| | | | 562, 589, 591 | | 725, 748, 750, 771, 772, 794, | 1062, 1063, 1064, |
| | | | | | 812, 815, 817, 836, 837, 860, | 1116, 1118, 1122, |
| | | | | | 861, 884, 885, 887, 889, 907, | 1124, 1171, 1172, |
| | | | | | 928, 929, 931, 933, 952, 973, | 1173, 1174, 1211 |
| | | | | | 975, 979, 996, 998, 1016 | |
| AT3G44670.1 | | 4, 56, 60, | 278, 426, 527, | 626, 627, 642 | 649, 651, 652, 653, 654, 656, | 984, 1005, 1006, |
| | | 237 | 570 | | 679, 680, 702, 725, 727, 747, | 1058, 1060, 1061, |
| | | | | | 748, 771, 772, 794, 813, 817, | 1062, 1064, 1069, |
| | | | | | 835, 838, 840, 860, 883, 884, | 1071, 1089, 1090, |
| | | | | | 906, 939, 941, 959, 980 | 1091, 1123, 1124, |
| | | | | | | 1125, 1126, 1170, |
| | | | | | | 1216 |
| AT3G46530.1 | RPP13 | | | 534 | 553, 554, 558, 573, 575, 576, | |
| | | | | | 595, 598, 599, 618, 619, 620, | |
| | | | | | 622, 624, 641, 684, 686, 689, | |
| | | | | | 711, 713, 716, 717, 742, 743, | |
| | | | | | 766, 767, 769, 788, 790, 792, | |
| | | | | | 793, 811, 814, 815 | |
| AT4G16860.1 | RPP4 | 4, 54, 162 | 198, 241, 378, | 509, 528, 529, | 558, 559, 562, 568, 591, 593, | 1114 |
| | | | 461, 473 | 530, 531 | 594, 616, 634, 638, 639, 641, | |
| | | | | | 661, 683, 705, 722, 726, 728, | |
| | | | | | 730, 748, 753, 755, 771, 773, | |
| | | | | | 775, 776, 794, 798, 799, 840, | |
| | | | | | 860, 862, 875, 894, 931, 951, | |
| | | | | | 953, 954, 956, 975, 1000, 1019, | |
| | | | | | 1021, 1066, 1069 | |
| AT4G16890.1 | SNC1 | 52, 92, 93, | 196, 239, 376, | 507, 526, 527, | 555, 556, 574, 576, 577, 599, | 1185, 1187, 1188, |
| | | 94, 96, 160 | 459, 471 | 528, 529 | 617, 621, 622, 624, 644, 686, | 1291, 1296 |
| | | | | | 706, 708, 712, 713, 714, 721, | |
| | | | | | 740, 777, 797, 799, 800, 802, | |
| | | | | | 821, 846, 855, 867, 1048, 1070, | |
| | | | | | 1089, 1091, 1136, 1139 | |
| AT4G16920.1 | | 4, 50, 90, | 194, 237, 374, | 505, 524, 525, | 553, 554, 557, 574, 576, 577, | 1187, 1191, 1294 |
| | | 91, 92, 94, | 457, 469 | 526, 527 | 599, 617, 621, 622, 624, 644, | |
| | | 158 | | | 666, 688, 705, 709, 711, 713, | |
| | | | | | 731, 736, 738, 754, 756, 758, | |
| | | | | | 759, 777, 781, 782, 823, 843, | |
| | | | | | 849, 850, 851, 858, 877, 914, | |
| | | | | | 934, 936, 937, 939, 958, 960, | |
| | | | | | 961, 983, 984, 1048, 1073, 1092, | |
| | | | | | 1094, 1139, 1142 | |
| AT4G16950.1 | RPP5 | 3, 53, 93, | 201, 243, 380, | 511, 530, 531, | 559, 560, 580, 582, 583, 605, | 1201, 1263, 1287, |
| | | 94, 95, 97, | 463, 475 | 532, 533 | 623, 627, 628, 630, 650, 672, | 1323, 1325, 1326, |
| | | 165 | | | 694, 711, 715, 717, 719, 737, | 1430, 1435 |
| | | | | | 742, 744, 760, 762, 764, 765, | |
| | | | | | 783, 787, 788, 829, 849, 855, | |
| | | | | | 857, 942, 943, 990, 1007, 1035, | |
| | | | | | 1079, 1098, 1100, 1145, 1148 | |
| AT5G38350.1 | | | 287, 291, 324 | 387 | 461, 463, 505, 521, 529, 531, | 764, 765, 772, |
| | | | | | 533, 620, 642, 661, 684, 685, | 780, 788, 816 |
| | | | | | 686 | |
| AT5G41740.1 | SSI4-LIKE | | 337, 435 | | 551, 552, 576, 587, 623, 639, | 914, 915, |
| | | | | | 641, 643, 644, 646, 668, 690, | 917, 1046 |
| | | | | | 707, 712, 729, 754, 777, 794, | |
| | | | | | 815, 818, 840 | |
| AT5G41750.1 | | | 346, 444 | | 590, 601, 637, 653, 655, 657, | 927, 928 |
| | | | | | 658, 660, 682, 704, 721, 726, | |
| | | | | | 743, 767, 790, 807, 828, 831, | |
| | | | | | 853 | |
| AT5G43470.1 | RPP8 | | 429, 440, 442, | | 558, 609, 610, 632, 658, 659, | |
| | | | 443, 481, 484, | | 678, 698, 700, 773, 776, 778, | |
| | | | 485, 486, 487, | | 779, 800, 802, 803, 804, 825, | |
| | | | 488 | | 875 | |
| AT5G43740.1 | | | 229, 467 | 519, 521 | 543, 545, 568, 569, 590, 615, | |
| | | | | | 617, 636, 637, 639, 688, 709, | |
| | | | | | 711, 712, 749, 751, 753, 772, | |
| | | | | | 774, 776, 777, 809, 811, 813, | |
| | | | | | 814, 833, 834 | |
| AT5G46510.1 | | 102 | 417 | 565 | 710, 724, 746, 748, 799, 801, | 1015, 1020, 1039, |
| | | | | | 826 | 1041, 1083, 1085 |
| AT5G46520.1 | VICTR-ACQOS | 103 | 418 | 566 | 711, 725, 747, 749, 800, 802, | 1016, 1021, 1040, |
| | | | | | 827 | 1042, 1084, 1086 |
| AT5G48620.1 | | | 429, 440, 442, | | 558, 609, 610, 632, 658, 659, | |
| | | | 443, 481, 484, | | 678, 698, 700, 773, 776, 778, | |
| | | | 485, 486, 487, | | 779, 800, 802, 803, 804, 825, | |
| | | | 488 | | 875 | |

All highly variable amino acids positions (>1.5 bit score) are based on reference accessions and are listed according to the protein region in which they are located.

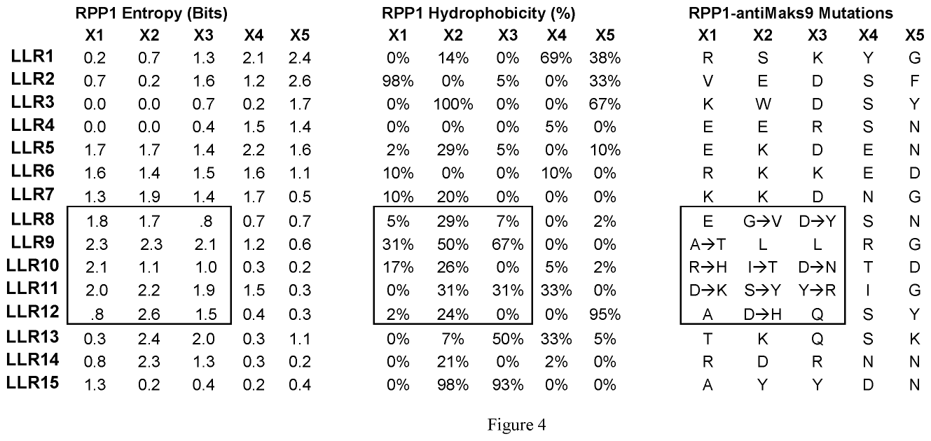

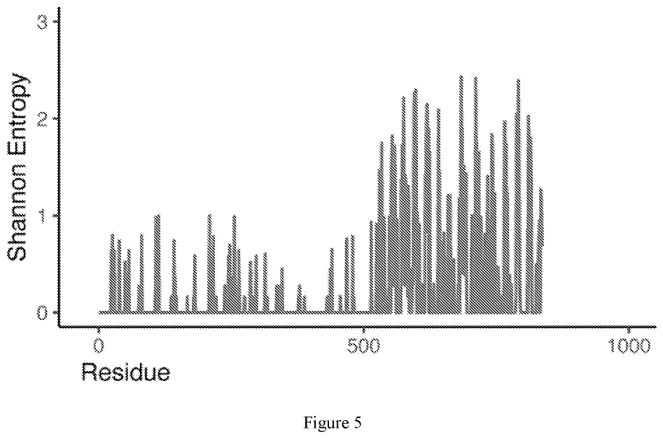

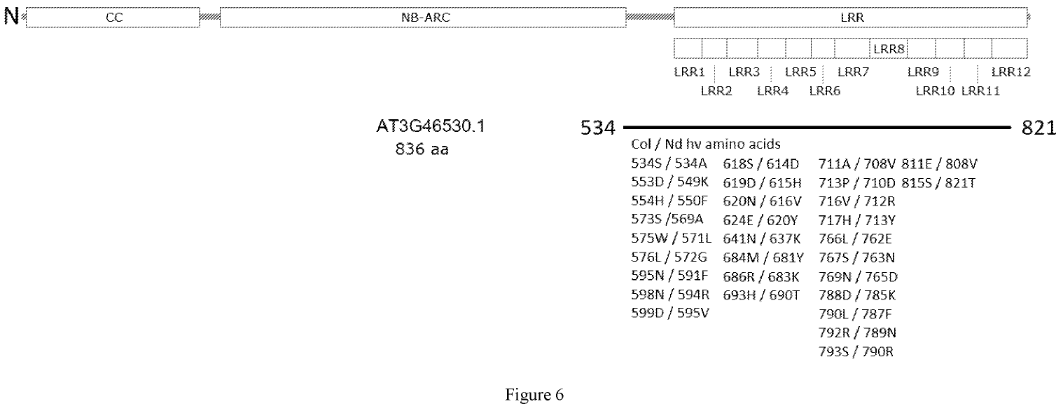

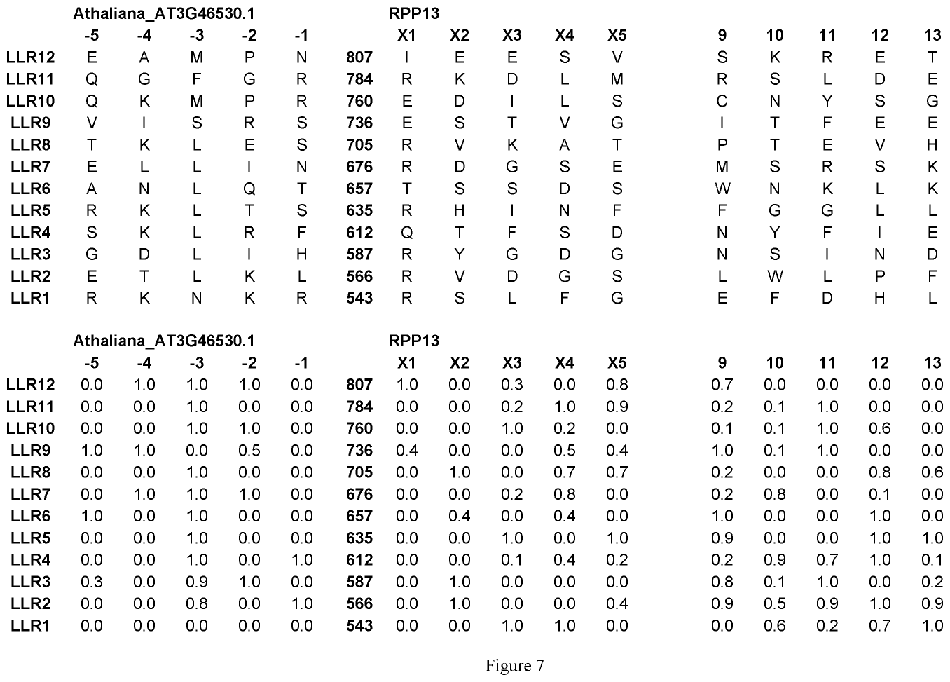

These highly variable amino acid residues may be modified in order to change the pathogen recognition, specificity, and/or binding of the given plant immunity receptor. FIG. 4 shows the entropy and hydrophobicity of LRRs in RPP1 of A. thaliana, and amino acid modifications that may be made to modify the pathogen recognition, specificity, and/or binding of the receptor. FIG. 5, FIG. 6, and FIG. 7 show the entropy and hydrophobicity of LRRs in RPP13 of A. thaliana, and amino acid modifications that may be made to modify the pathogen recognition, specificity, and/or binding of the receptor.

Surface-Exposed Highly Variable Residues Help Predict hvNLR Binding Sites

The variability scores from the alignment of NLR clades afford a residue-resolution view of cross-species diversity of the NLR immune receptors. This information was combined with structure modelling and residue composition analysis to predict ligand-binding sites on the surfaces of highly variable LRR domains. Because the concave side of LRR domains contains a beta-sheet with a regular array of surface exposed residues, it can be represented as a table with one line per repeat unit and the columns corresponding to variable positions in the canonical LXXLXLXX repeat. The representations were used to analyze both sequence variability and residue type composition of RPP1, RPP13, and Sr33. Both the entropy and percent hydrophobic residues over the alignment varied among the surface residues in the NLR. However, there were residues that were both high variable and hydrophobic. Therefore, regions of 10-15 contiguous residues having both highly variable and hydrophobic residues were selected in each receptor to determine whether amino acid modifications therein impact pathogen recognition and binding.

Agrobacterium-mediated transient expression assays in N. benthamiana and protoplast co-transfection assays in Triticum aestivum in the art were used to test the activity of the engineered alleles.

### De Novo Identification of New Immune Receptors

As demonstrated herein, Shannon entropy may be used to analyze plant immune receptors, e.g., predict their binding sites, and predict the receptors that are highly variable. Because innate immune receptors exhibit high variability, Shannon entropy analysis may be used for de novo identification of plant immune receptors. For example, the genomes of a given set of plants may be subjected to Shannon entropy analysis. Sequences that exhibit high variability may be further analyzed using methods in the art to determine if they are part of a gene that encodes a receptor-like protein.

The following examples are intended to illustrate but not to limit the invention.

### Examples

Sequences

Sr33 (Uniprot S5DII7_WHEAT, Accession No. AGQ17390.1):
MDIVTGAIAKLIPKLGELLVGEYKLHKGVKKNIEDLLKELKTMNAALIKIGEVPPDQLDSQDKLW
ADEVRELSYVIEDAVDKFLVRVHGVEPDDNTNGFKGLMKRTTKLLKKVVDKHGIAHAIKDIKKEL
QEVAARRDRNKFDGIASIPTEAIDPRLRALYIEAAELVGIYGKRDQELMSLLSLEGDDASTKKLK
KVSIVGFGGLGKTTLAKAVYEKIKGDFDCHAFVPVGQNPDKKKVFRDILMDLSNSNSDLALLDER
QLINKLHKFLENKRYLVIIDDVWDEGLWKDINLAFSNRNNLGSRLIITTRIFGVSESCCSSADDP
VYEIEPLSIDDSSKLFYTRIFSDSGCPKEFEQVSKDILKKCGGVSLAIITIASALASGQQVKPKH
EWDILLQSLGSGVTKDNSLVEMRRILSFSYYNLPSHLKTCLLYLCIYPEDSMIHRDRLIWKWVAE
GFVHHGDQGTSLFLVGLNYFNQLINRSMLQPIYSDMGNVYACRVHDMVLDLICNLSHEAKFVNVF
DGTGNIMSSQSNVRRLSLQNKNEDHQAKPLTNIMSISQVRSITIFPPAVSIMPALSRFEVLRVLD
LSDCNLGESSSLQPNLKGVGHLIHLRYLGLSGTRISKLPAEIGTLQFLEVLDLGYNHELDELPST
LFKLRRLIYLNVSPYKVVPTPGVLQNMTSIEVLRGIFVSLNIIAQELGKLARLRELQIYFKDGSL
DLYEGFVKSLCNLHHIESLIVSCNSGETSFELMDLLGEQWVPPVHLREFVSEMPSQLSALRGWIK
RDPSHLSNLSELILPTVKEVQQEDVEIIGGLLSLRRLLIESTHQTQRLLVIRADGFRCMVDFYLN
CGSATQIMFESGALPRAEEVCFSLGVRVAKEDGNRGFDLGLQGNLLSLRRVVWVKMYCGGARVGE
AKEAKAAVRHALEDHPNHPPIQINMFPRIAEGAQDDDLMCYPVGGPISDAE (SEQ ID NO:
1)
Sr50 (Uniprot A0A0S2LJ10_SECCE, Accession No. ALO61074.1):
MNIVTGAMGSLIPKLGELLMDEYKLHKRIKKDVEFLKKELESMHAALIKVGEVPRDQLDRQVKLW
ADEVRELSYNMEDVVDKFLVRVDGDGIQQPHDNSGRFKELKNKMIGLFKKGRNHHRIADAIKEIK
EQLQEVAARRDRNKVAVPNPMEPITIDPCLRALYAEATELVGIYGKRDEELMRLLSMEGDDASNK
RLKKVSIVGFGGLGKTTLARAVYDKIKGDFDCRAFVPVGQNPDMKKVLRDILIDLGNPHSDLAIL
DDKQLVKKLHDFLENKRYLVIIDDIWDEMLWEGINFAFSNRNNLGSRLITTTRNFDVSKSCCLSA
DDSIYKMKPLSTDDSRRLFHKRIFPDAGGCPSEFQQVSEDILKKCGGVPLAIITIASALASGQHV
KPKHEWDILLQSLGSGVTKDNSLVEMRRILSFSYYNLPSHLKTCLLYLCIYPEDSTIGRDRLIWK
WVAEGFVHHGDQGTSLFLVGLNYFNQLINRSMIQPIYDELGQVHACRVHDMVLDLICNFSHEAKF
VNVLDGTGNSISSQSNVRRLSLQNKMEDHQAKPLTNIMSMSRVRSITIFPPAVSIMPSLSMFEVL
RVLDLSNCDLGKSSSLQLNLKGVGHLIHLRYLDLQGTQISELPTEIGNLQFLEVLDLDNNYELDE
LPSTLFKLRRLIYLNVMLYKVVPTPGVLQNMTSIEVLRGVLVSLNIIAQELGNLTRLRELKICFK
DGNLDSYKLFVKSLGNLHHIESLSISYNSKETSFELMDLLGERWVPPVHLREFVSWMPSQLSALR
GWIKRDPSHLSNLSELILWPVKEVQQEDVEIIGGLLSLRRLWIKSTHQTQRLLVIRADGFRCMMD
FELNCGSAAQIMFEPGALPRAEVLVFSLGVRVAQEDGNCGFDLGLQGNLLSLRHDVFVRIYCGGA
RVGEAKEAEAAVRHALEAHPNHPPIDIEMTPYIAEGARDDDLCEEN (SEQ ID NO: 2)
Full length AvrSr50 sequence as reported in Chen et al. (2017):
MMHSIIFQTLLIITIVFSKVWGARSLVKIDWSGSEYTILGANHYEEPNTGAAAQFPGTMTVDDGR
SPYIVRKLRNSSGKRFYVFTGHPQQPIVWNPHEEIEIQFNRKFLIAVLTEFEADSQVFNHFARRQ
HR (SEQ ID NO: 3)
Cloned AvrSr50 (without signal peptide):
MARSLVKIDWSGSEYTILGANHYEEPNTGAAAQFPGTMTVDDGRSPYIVRKLRNSSGKRFYVFTG
HPQQPIVWNPHEEIEIQFNRKFLIAVLTEFEADSQVFNHFARRQHR (SEQ ID NO: 4)

### Sr50 Binding Site Prediction

Allelic diversity of the wheat stem rust resistance gene Sr50 was gathered by blast from nr database using Sr50 protein sequence (Accession No. ALO61074.1) as a query. The top 100 hits were aligned full length using PRANK (Löytynoja 2014) and the alignment was edited in Jalview to adjust mis-aligned columns and to remove poorly aligned sequences, resulting in an alignment of 93 sequences. The entropy scores were generated using the Protein Variability Server (imed.med.ucm.es/PVS/). The structural model of Sr50 LRR domain was predicted using PHYRE (Kelley et al. 2015). In Sr50 sequence, 16 LRR motifs were annotated manually and entropy scores as well as hydrophobicity scores for the individual strands of LRRs (LxxLxLxx) were presented in a tabular format. Resulting data was examined for clustering of residues that showed both high entropy scores and the presence of hydrophobic residues resulting in strands 8-14 predicted as the center of the binding site.

Sr3350 Allelic Swap and Transient Co-Expression with AvrSr50 in Nicotiana benthamiana

The chimeric protein Sr3350 was constructed by grafting Sr50 leucine-rich repeats residues 8 to 14 onto its ortholog Sr33 (S5DII7_WHEAT) using unique restriction digest sites (BbvCI and SbfI). The binary vector p1776 containing each gene (Sr50, Sr33, Sr3350) was transformed into Agrobacterium strain GV3101 pMP90 by electroporation and inoculated in 4-5 week-old N. benthamianan plants at an OD₆₀₀ of 0.025 with or without the pathogen effector AvrSr50 or its variant race AvrSr50QCMJC (Chen et al. 2017). Four experimental repeats were performed for each experiment on different plants. Phenotypes of the hypersensitive response were recorded two days post infiltration.

Sr3350 Allelic Swap and Cell Death Assays with AvrSr50 in Triticum aestivum (Wheat) Protoplasts

The chimeric Sr3350 construct generated as described above was domesticated and cloned using the Golden Gate cloning method (Engler et al., 2014, ACS Synthetic Biology) into the level 0 acceptor pAGM1287. The genes Sr33 and Sr50 were also domesticated to remove internal restriction sites for the T3 S enzymes Eco31I (BsaI) and BpiI (BbsI) (Thermo Fisher Scientific Waltham, Mass., USA) that are used in the Golden Gate method and cloned into the same vector. Once the correct sequence was confirmed via sequencing the constructs were taken further to the level 1 acceptor pICH47803 with the Zea maize Ubiquitin promoter, a 3×FLAG tag, and the Nos (nopaline synthase) terminator from Agrobacterium tumefaciens.

For isolation of protoplasts, wheat seeds were grown for two weeks on filter paper under sterile conditions. Once seedlings showed two leaves the epidermis of the leaves was peeled off by making a shallow cut with a razor blade on the abaxial side of the leaf. Peeled leaves were incubated in solution for 3 hours to release wheat protoplasts in the solution. Wheat protoplasts were washed, counted and transfected using PEG4000 (Sigma-Aldrich, St. Louis, Mo.) as described in Saur et al., 2019 Plant Methods 15; 118. After transfection protoplasts were incubated in 12-well plates for two days. After two days the protoplasts were lysed in cell culture lysis buffer (Promega Luciferase Assay System, Catalog number E4550, Promega, San Luis Obispo, Calif.) and transferred to a 96-well cell culture plate (Greiner Bio-One Catalog Number 655075, Greiner Bio-One, Kremsmünster, Austria). The Luciferin substrate (Promega, San Luis Obispo, Calif., see above) was added to each well with a multi-channel pipette and the luminescence was measured in a 96-well plate reader (TECAN Infinite F Plex; 20 minutes, no attenuation).

### Data Sources

Arabidopsis pan-NLRome nucleotide assemblies were downloaded from the 2blades foundation (2blades.org/resources/). Gene annotations were downloaded from GitHub pan-NLRome repository (github.com/weigelworld/pan-nlrome/). The gene models that matched assemblies were available for 62 A. thaliana accessions (Van de Weyer et al. 2019), and these were processed to extract the amino acid sequences of captured protein-coding genes using bedtools getfasta program (Quinlan 2014). The reference set of 168 NLR proteins of the Arabidopsis Col-0 genome was extracted using methods in the art. See, e.g., Sarris et al. 2016.

### NLRome Phylogenetic Analysis and Clade Assignments

Phylogenetic tree construction for the whole A. thaliana NLRome was performed using methods in the art. See, e.g., Bailey et al. 2018. Briefly, amino acid sequences were searched for the presence of NB-ARC domain using hmmsearch and an extended NB-ARC Hidden Markov Model (HMI) 13059_2018_1392_MOESM16_ESM.hmm and initial alignment was made on this HMI using −A option. The resulting alignment was processed to convert unaligned characters to gaps using ‘tr a-z-’ command, and reformatted with ‘esl-reformat—migap’ to remove all gaps followed by ‘esl-alimanip—lmin 237’ command to retained aligned sequences that matched at least 70% of the HMM model. The filtered alignment as well as the alignment not-filtered by HMM coverage were used to construct maximum likelihood phylogenies using raxml (raxml-T 8-n Raxml.out-f a-x 12345-p 12345-#100-m PROTCATJTT-s NLRome.HMMhits.Matches.237 min.fa), which contained 7,818 leaves at 70% HMI coverage and 11,078 leaves, not filtered by HMM coverage. The trees were visualized in iToL. The phylogeny filtered by coverage was used to assign CNL and RNL protein sequences into clades, while the unfiltered phylogeny was used for TNL clade assignment as some clades of TNLs did not contain complete NB-ARCs. To define clades, we first used branch length of <0.9 within clade >99% bootstrap replicate support followed by grouping remaining sequences based on manual analysis of the phylogeny. Lists of genes from each clade were exported from iToL.

### Sequence Alignment and Variability Analysis

For each identified clade, full length protein sequences were aligned using PRANK algorithm using methods in the art. See, e.g., Löytynoja 2014. The resulting alignments were imported into R and entropy plots were created for individual subclades within RNL, TNL, CNL, NLR-ID and PairedHelper clades using R codes deposited at github.com/krasileva-group/hvNLR. The entropy plots were examined for the presence of a strong baseline (invariant residues). If a baseline was at 0, the clade was not split further, if the baseline was higher indicating the presence of multiple paralogous groups of genes, the clade was passed on to raxml phylogeny construction and sub-clades were constructed as described above. This process was iterated 2-3 times resulting in the final assignment of all genes into sub-clades and construction of final sets of entropy plots. The entropy plots were visually examined and alignments that consistently exceeded 1.5 bits in the LRR regions while maintaining baseline at 0 were marked as highly variable.

Binding Site Prediction in hvNLRs

All highly variable clades were examined for the presence of Arabidopsis Col-0 allele. For these Col-0 alleles, lists of 15 amino acid positions with the highest entropy scores (Table 1) and lists of amino acid positions corresponding to all highly variable residues (bit score >1.5) (Table 2) were extracted using HvGenes.R script (github.com/krasileva-group/hvNLR). The second script HvClades.R was used to map entropy scores to the predicted concave surface of the LRR domain. The entropy scores for the individual strands of LRRs (LxxLxLxx) were exported in tabular format. The hydrophobicity scores for these residues were calculated as the percent of hydrophobic residues at a given amino acid position and exported as a second table. Resulting 2D representations of entropy and hydrophobicity of the concave sides were visually examined for clustering of residues that showed both high entropy scores and the presence of hydrophobic residues.

Experiments with Other Plant Species

The methods described herein were used to identify hvNLRs and highly variable amino acid residues in: Brachypodium distachyon (model grass species), Zea maize (maize), Glycine max (soybean).

Screening Modifications of hvNLRs to Derive New Specificities

The methods described herein may be used to design libraries of modified plant immunity receptors. For example, libraries in which the amino acid sites of a given hvNLR are randomized to create up to about 10⁸ variants. The libraries may be screened for a desired activity and a given plant can then be engineered to express the variant immunity receptor that has the desired activity using methods in the art. For example, selected sets of amino acid residues that form a putative ligand binding site may be used for modifications. Library synthesis can be achieved by targeting short regions corresponding to 4-6 LRR repeats containing predicted binding sites and 10-15 top scoring highly variable amino acid residues (bit score >1.5) in each region. The library may be screened, e.g., by using a yeast protein binding system, for binding to a given effector protein that is derived from a pathogen of interest. The activity of the hvNLRs may be evaluated in Nicotiana benthamiana and protoplast systems as described above. Plants of interest may be modified to express one or more of the hvNLR variants that bind the given effector protein. The modified plants that exhibit resistance against the pathogen may be further modified and/or propagated.

### REFERENCES

The following references are herein incorporated by reference in their entirety with the exception that, should the scope and meaning of a term conflict with a definition explicitly set forth herein, the definition explicitly set forth herein controls:
Bailey, et al. (2018) Dominant Integration Locus Drives Continuous Diversification of Plant Immune Receptors with Exogenous Domain Fusions. Genome Biology 19 (1): 23.
Chen, et al. (2017) Loss of by Somatic Exchange in Stem Rust Leads to Virulence for Resistance in Wheat. Science 358 (6370): 1607-10.
Han, et al. (2008) Antigen recognition by variable lymphocyte receptors. Science. 2008 Sep. 26; 321(5897):1834-7.
Jehl, et al. (2015) OD-seq: outlier detection in multiple sequence alignments. BMC Bioinformatics. 16, 269-11.
Kelley, et al. (2015) The Phyre2 Web Portal for Protein Modeling, Prediction and Analysis. Nature Protocols 10 (6): 845-58.
Lagudah, E., Periyannan, S. K., Scientific, C., and Organisation, I. R. (2014) Wheat stem rust resistance gene.
Lagudah, E., Periyannan, S., Steuernagel, B., Witek, K., Wulff, B., Two Blades Foundation, Scientific, C., and Organisation, I. R. (2017) Wheat stem rust resistance genes and methods of use.
Lagudah, E., Spielmeyer, W., Keller, B., Krattinger, S., Scientific, C., Industrial Research Organization (CSIRO), Research, G., Corp, D., University of Zurich (2011) Resistance genes.
Löytynoja, Ari. (2014) Phylogeny-Aware Alignment with PRANK. Methods in Molecular Biology 1079: 155-70.
Periyannan, S. K., Dodds, P. N., Mago, R., Lagudah, E., Scientific, C., and Organisation, I. R. (2017) Stem rust resistance gene.
Quinlan A R. (2014) BEDTools: The Swiss-Army Tool for Genome Feature Analysis. Curr Protoc Bioinformatics. 47:11.12.1-34.
Sarris, et al. (2016) Comparative Analysis of Plant Immune Receptor Architectures Uncovers Host Proteins Likely Targeted by Pathogens. BMC Biology 14 (February): 8.
Saur, et al. (2019) A cell death assay in barley and wheat protoplasts for identification and validation of matching pathogen AVR effector and plant NLR immune receptors. Plant Methods 15, 118.
Shannon & Weaver (1949) The Mathematical Theory of Communication, Univ. of Illinois Press. ISBN 0-252-72548-4.
Slater & Birney (2005) Automated generation of heuristics for biological sequence comparison. BMC Bioinformatics. 6, 31-11.
Stewart, et al. (1997) A Shannon entropy analysis of immunoglobulin and T cell receptor. Molec Immunol 34(15): 1067-1082.
Van de Weyer, et al. (2019) A Species-Wide Inventory of NLR Genes and Alleles in Arabidopsis Thaliana. Cell 178 (5): 1260-72.e14.
U.S. Ser. No. 10/113,180B2
WO2014194371A1
WO2017024053A1
WO2017091847A1

All scientific and technical terms used in this application have meanings commonly used in the art unless otherwise specified.

The sequences identified by accession numbers and/or Uniprot numbers are herein incorporated by reference in their entirety.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109. As used herein, an amino acid or nucleotide of a given sequence that “corresponds” to an amino acid or nucleotide of a reference sequence refers to the amino acid or nucleotide of the given sequence that aligns with the amino acid or nucleotide of the reference sequence when the given sequence and the reference sequence are optimally aligned.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus.

Polypeptides may be made using methods known in the art including chemical synthesis, biosynthesis or in vitro synthesis using recombinant DNA methods, and solid phase synthesis. See, e.g., Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, Ill., which are herein incorporated by reference. Polypeptides may be purified using protein purification techniques known in the art such as reverse phase high-performance liquid chromatography (HPLC), ion-exchange or immunoaffinity chromatography, filtration or size exclusion, or electrophoresis. See, e.g., Olsnes and Pihl (1973) Biochem. 12(16):3121-3126; and Scopes (1982) Protein Purification, Springer-Verlag, NY, which are herein incorporated by reference. Alternatively, the polypeptides may be made by recombinant DNA techniques known in the art. Thus, polynucleotides that encode the polypeptides described herein are contemplated. In some embodiments, the polypeptides and polynucleotides are isolated.

As used herein, an “isolated” compound refers to a compound that is isolated from its native environment. For example, an isolated polynucleotide is a one which does not have the bases normally flanking the 5′ end and/or the 3′ end of the polynucleotide as it is found in nature. As another example, an isolated protein fragment is a one which does not have its native amino acids, which correspond to the full-length polypeptide, flanking the N-terminus, C-terminus, or both.

As used herein, a compound (e.g., receptor) “specifically binds” a given target (e.g., ligand or epitope) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e.g., a given epitope). As used herein, an “epitope” is the part of a molecule that is recognized by, e.g., a receptor. Epitopes may be linear epitopes or three-dimensional epitopes. As used herein, the terms “linear epitope” and “sequential epitope” are used interchangeably to refer to a primary structure, e.g., a linear sequence of consecutive amino acid residues, that is recognized by, e.g., a receptor. As used herein, the terms “three-dimensional epitope” and “conformational epitope” are used interchangeably to refer a three-dimensional structure, e.g., a plurality of amino acid residues, which need not be consecutive, that together form an epitope.

As used herein, “binding affinity” refers to the propensity of a compound to associate with (or alternatively dissociate from) a given target and may be expressed in terms of its dissociation constant, Kd. In some embodiments, the antibodies have a Kd of 10⁻⁵ or less, 10⁻⁶ or less, preferably 10⁻⁷ or less, more preferably 10⁻⁸ or less, even more preferably 10⁻⁹ or less, and most preferably 10⁻¹⁰ or less, to their given target. Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative.

Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As provided herein, amino acid modifications are indicated by the amino acid residue (or residues) and their amino acid position based on the parental polypeptide (i.e., the wildtype or unmutated polypeptide) followed by the specific modification. Amino acid “modifications” of a given amino acid residue include amino acid substitutions (including non-canonical amino acids), post-translational modifications, chemical modifications, deletion of the given amino acid, and addition of an amino acid before, on, or after the given amino acid. As provided herein, preferred amino acid modifications are amino acid substitutions.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of modifying pathogen recognition, binding, and/or specificity of a plant immunity receptor, which comprises selecting one or more amino acid residues in the plant immunity receptor that will be modified by
determining the Shannon entropy of each amino acid of the plant immunity receptor;
selecting an entropy cutoff value of at least 1.0, at least 1.1, at least 1.2, at least 1.3, at least 1.4, at least 1.5, at least 1.6, at least 1.7, at least 1.8, at least 1.9, or at least 2.0; and
selecting at least one amino acid residue amino acid residues among the amino acid residues having the highest entropy values above the entropy cutoff value.

2. A method of modifying pathogen recognition, binding, and/or specificity of a plant immunity receptor, which comprises
modifying (e.g., substituting, deleting, or adding an amino acid before or after) one or more amino acid residues that are identified as having the highest entropy values above the entropy cutoff value.

3. The method according to claim 2, wherein the Shannon entropy is determined from a plurality of amino acid sequences from homologs of the plant immunity receptor, and wherein the plant immunity receptor and the homologs are of the same plant species.

4. The method according to claim 3, wherein the plurality comprises at least 25, at least 30, at least 35, at least 40, at least 45, at least 50, at least 55, at least 60, at least 65, at least 70, at least 75, or at least 80 amino acid sequences.

5. The method according to claim 3, wherein the plants are genetically diverse.

6. The method according to claim 2, wherein at least ten amino acid residues having the highest entropy values are identified as being likely involved in binding the cognate ligand.

7. The method according to claim 2, wherein 5-20, 6-19, 7-18, 8-16, 9-16, or 10-15 amino acid residues having the highest entropy values are identified as being likely involved in binding the cognate ligand.

8. The method according to claim 2, and further comprising determining the hydrophobicity of each amino acid of the receptor and identifying one or more of the amino acid residues having the highest entropy values and the highest hydrophobicity as being likely involved in binding the cognate ligand.

9. A method of modifying pathogen recognition, binding, and/or specificity of a plant immunity receptor, which comprises modifying one or more amino acid residues that are identified as being involved in binding the cognate ligand of the plant immunity receptor according to claim 2.

10. The method according to claim 9, wherein the one or more amino acid residues are substituted with a different amino acid.

11. The method according to claim 9, wherein a region of contiguous amino acid residues comprising the one or more amino acid residues is substituted with a region of contiguous amino acid residues obtained from a different plant immunity receptor.

12. A method of modifying a Sr33 plant immunity receptor, which comprises modifying one or more of the amino acid residues that correspond to amino acid residue positions 703-887 of SEQ ID NO: 1.

13. The method according to claim 12, wherein the amino acid residue positions are selected from
(a) 707, 709, 710, 735, 737, 738, 765, 767, 768, 793, 795, 796, 818, 820, and 821 of SEQ ID NO: 1, or
(b) 707, 709, 711, 735, 737, 741, 765, 767, 793, 795, 796, 818, 820, 821, 832, 841, 843, 864, 866, and 880 of SEQ ID NO: 1.

14. The method of claim 2, wherein the plant immunity receptor is of Arabidopsis thaliana, and the one or more amino acid residues that are modified correspond the highly variable amino acid residues as set forth in Table 1 or Table 2.

15. A plant immunity receptor made by the method according to claim 9.

16. A nucleic acid molecule which encodes the plant immunity receptor according to claim 15.

17. A vector comprising the nucleic acid sequence of claim 16.

18. A cell comprising the vector of claim 17.

19. A plant comprising the plant immunity receptor of claim 15.

20. A population of plants comprising a plurality of plants according to claim 19.


SEQUENCE LISTING (4 sequences)

SEQ ID NO: 1 - protein, 961 aa, Triticum aestivum
MDIVTGAIAKLIPKLGELLVGEYKLHKGVKKNIEDLLKELKTMNAALIKIGEVPPDQLDSQDKLWADEVRELSYVIEDAVDKFLVRVHGVEPDDNTNGFKGLMKRTTKLLKKVVDKHGIAHAIKDIKKELQEVAARRDRNKFDGIASIPTEAIDPRLRALYIEAAELVGIYGKRDQELMSLLSLEGDDASTKKLKKVSIVGFGGLGKTTLAKAVYEKIKGDFDCHAFVPVGQNPDKKKVFRDILMDLSNSNSDLALLDERQLINKLHKFLENKRYLVIIDDVWDEGLWKDINLAFSNRNNLGSRLIITTRIFGVSESCCSSADDPVYEIEPLSIDDSSKLFYTRIFSDSGCPKEFEQVSKDILKKCGGVSLAIITIASALASGQQVKPKHEWDILLQSLGSGVTKDNSLVEMRRILSFSYYNLPSHLKTCLLYLCIYPEDSMIHRDRLIWKWVAEGFVHHGDQGTSLFLVGLNYFNQLINRSMLQPIYSDMGNVYACRVHDMVLDLICNLSHEAKFVNVFDGTGNIMSSQSNVRRLSLQNKNEDHQAKPLTNIMSISQVRSITIFPPAVSIMPALSRFEVLRVLDLSDCNLGESSSLQPNLKGVGHLIHLRYLGLSGTRISKLPAEIGTLQFLEVLDLGYNHELDELPSTLFKLRRLIYLNVSPYKVVPTPGVLQNMTSIEVLRGIFVSLNIIAQELGKLARLRELQIYFKDGSLDLYEGFVKSLCNLHHIESLIVSCNSGETSFELMDLLGEQWVPPVHLREFVSEMPSQLSALRGWIKRDPSHLSNLSELILPTVKEVQQEDVEIIGGLLSLRRLLIESTHQTQRLLVIRADGFRCMVDFYLNCGSATQIMFESGALPRAEEVCFSLGVRVAKEDGNRGFDLGLQGNLLSLRRVVWVKMYCGGARVGEAKEAKAAVRHALEDHPNHPPIQINMFPRIAEGAQDDDLMCYPVGGPISDAE

SEQ ID NO: 2 - protein, 956 aa, Secale cereale
MNIVTGAMGSLIPKLGELLMDEYKLHKRIKKDVEFLKKELESMHAALIKVGEVPRDQLDRQVKLWADEVRELSYNMEDVVDKFLVRVDGDGIQQPHDNSGRFKELKNKMIGLFKKGRNHHRIADAIKEIKEQLQEVAARRDRNKVAVPNPMEPITIDPCLRALYAEATELVGIYGKRDEELMRLLSMEGDDASNKRLKKVSIVGFGGLGKTTLARAVYDKIKGDFDCRAFVPVGQNPDMKKVLRDILIDLGNPHSDLAILDDKQLVKKLHDFLENKRYLVIIDDIWDEMLWEGINFAFSNRNNLGSRLITTTRNFDVSKSCCLSADDSIYKMKPLSTDDSRRLFHKRIFPDAGGCPSEFQQVSEDILKKCGGVPLAIITIASALASGQHVKPKHEWDILLQSLGSGVTKDNSLVEMRRILSFSYYNLPSHLKTCLLYLCIYPEDSTIGRDRLIWKWVAEGFVHHGDQGTSLFLVGLNYFNQLINRSMIQPIYDELGQVHACRVHDMVLDLICNFSHEAKFVNVLDGTGNSISSQSNVRRLSLQNKMEDHQAKPLTNIMSMSRVRSITIFPPAVSIMPSLSMFEVLRVLDLSNCDLGKSSSLQLNLKGVGHLIHLRYLDLQGTQISELPTEIGNLQFLEVLDLDNNYELDELPSTLFKLRRLIYLNVMLYKVVPTPGVLQNMTSIEVLRGVLVSLNIIAQELGNLTRLRELKICFKDGNLDSYKLFVKSLGNLHHIESLSISYNSKETSFELMDLLGERWVPPVHLREFVSWMPSQLSALRGWIKRDPSHLSNLSELILWPVKEVQQEDVEIIGGLLSLRRLWIKSTHQTQRLLVIRADGFRCMMDFELNCGSAAQIMFEPGALPRAEVLVFSLGVRVAQEDGNCGFDLGLQGNLLSLRHDVFVRIYCGGARVGEAKEAEAAVRHALEAHPNHPPIDIEMTPYIAEGARDDDLCEEN

SEQ ID NO: 3 - protein, 132 aa, Artificial Sequence
  Full length AvrSr50 sequence as reported in Chen et al. (2017) Loss of by Somatic Exchange in Stem Rust Leads to Virulence for Resistance in Wheat. Science 358 (6370) 1607-10.
MMHSIIFQTLLIITIVFSKVWGARSLVKIDWSGSEYTILGANHYEEPNTGAAAQFPGTMTVDDGRSPYIVRKLRNSSGKRFYVFTGHPQQPIVWNPHEEIEIQFNRKFLIAVLTEFEADSQVFNHFARRQHR

SEQ ID NO: 4 - protein, 111 aa, Artificial Sequence
  Cloned AvrSr50 sequence without signal peptide.
MARSLVKIDWSGSEYTILGANHYEEPNTGAAAQFPGTMTVDDGRSPYIVRKLRNSSGKRFYVFTGHPQQPIVWNPHEEIEIQFNRKFLIAVLTEFEADSQVFNHFARRQHR